(19) United States
(12) Patent Application Publication
(10) Pub. No.: US 2020/0102426 A1
(43) Pub. Date: Apr. 2, 2020

(54) SCALE PRODUCTION PROCESS OF PURIFIED BACTERIAL CELLULOSE HYDROGEL OBTAINED BY THE POLYMERIZATION OF GLUCOSE FROM SUGARS OF RENEWABLE SOURCES VIA BIOTECHNOLOGY BY THE SPREAD OF CELLULOSE-PRODUCING BACTERIA, PARTICULARLY GLUCONOACETOBACTER HANSENII LMSPE, IN REACTORS FOR APPLICATION IN THE HEALTH, PHARMACOTECHNICAL AND COSMETRY AREAS

(71) Applicant: POLISA BIOPOLIMEROS PARA SAUDE LTDA - EPP, Recife (BR)

(72) Inventors: Francisco de Assis Dutra Melo, Recife (BR); Lara Moura Aguiar, Recife (BR); Daniel Moura Aguiar, Recife (BR)

(73) Assignee: POLISA BIOPOLIMEROS PARA SAUDE LTDA - EPP, Recife (BR)

(21) Appl. No.: 16/145,548

(22) Filed: Sep. 28, 2018

(51) Int. Cl. C08J 3/075; C08L 1/02; C12N 1/20

(52) U.S. Cl. CPC C08J 3/075; C08L 1/02; C08L 2203/02; C08J 2301/02; C12N 1/20

(57) ABSTRACT

A scale production process of purified bacterial cellulose hydrogel obtained by the polymerization of glucose from sugars of renewable sources via biotechnology by the spread of cellulose-producing bacteria, particularly gluconoacetobacter hansenii lmspe, in reactors for application in the health, pharmacotechnical and cosmetry areas, which provides for the steps of a) hydration of the purified bacterial cellulose, structured in the micro- and nano-fibrillar fractions; b) homogenization of purified bacterial cellulose structured in the micro- and nano-fibrillar fractions; c) obtaining the hydrogel with the two fractions; d) hydrogel filtration; e) obtaining two products: 1. Nanocellulose hydrogel; 2. Microcellulose hydrogel; the scale production process of two fractions of bacterial cellulose hydrogel, 1. Nanocellulose hydrogel; 2. Microcellulose hydrogel is obtained in blender units for the homogenization of the hydrated cellulose and for the separation of the hydrogels by centrifugal filtration.

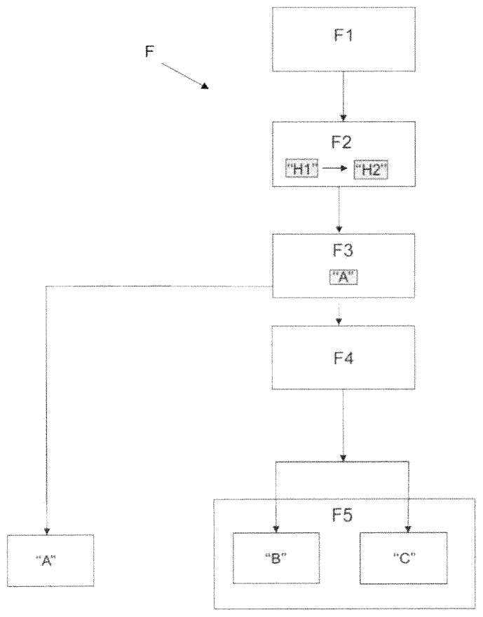

### BACKGROUND OF THE DISCLOSURE

### 1. Field of the Disclosure

This patent application focuses on the scale production of bacterial cellulose hydrogel, which is intended for the development of products for application in the fields of activities that involve health, pharmacotechnics and cosmiatry.

This specification deals with a patent application that proposes a process to produce, in scale, nano-fibrillar and micro-fibrillar bacterial cellulose hydrogel, obtained through the polymerization of glucose based on sugars from renewable sources, through the use of biotechnology, through the propagation of cellulose-producing microorganisms, such process allowing the hydrogel and products resulting therefrom to have particular use in the fields of health (medicine), pharmacotechnics and cosmiatry.

### 2. Description of the Related Art

Processes developed for the production of hydrogel from polymers and copolymers from natural and synthetic sources are known from the prior art, intended for in natura application and as a matrix for the production of films, membranes and composites aimed at applications in the fields of health (medicine), pharmacotechnics and cosmiatry.

It is essential that the processes for the production of bacterial celluloses hydrogel and its products must meet, in relation to the characteristics of the product obtained, strict criteria in relation to its purity, physical characteristics of micro-crystallinity and micro-fibrillar and nano-fibrillar structure of cellulose and meet the production scale.

The hydrogels currently available and which belong, as a consequence, to the current state of the art and which are intended for application in the medical areas are composed of a dispersing phase of biological origin such as hyaluronic acid with synthetic microparticles in suspension.

Hydrogels associated with synthetic polymers and also incorporating the prior art are biocompatible, but do not develop biointegration and remodeling, occurring their elimination, over time, therefore they are not suitable for application in medicine.

On the other hand, hydrogels of biological origin produced by the hydration of copolymers such as collagen and chitosan derivatives are obtained from animals and present the risk of contamination. These copolymers contain in their structure amino groups which give them potential risk of rejection. In addition, with the exception of collagen, they have not yet reached an adequate scale production process.

Bacterial cellulose is a biopolymer, obtained by microbial synthesis, consisting of a complex of cellulose filaments of nano- and micro-fibrillar structure, with high purity. Its chemical structure consists of 97.8% of polymerized glucose from sugars from renewable sources such as sugar cane that can guarantee its production in scale with quality control.

Its chemical composition consisting essentially of glucose allows its application in medicine without the risk of developing rejection. The use of bacterial cellulose in its original form of synthesis consisting of a complex of nano- and micro-fibrils of cellulose is biocompatible and nontoxic and meets with great efficiency the demand of dressings in the form of membranes as a protective mechanical barrier in epithelial lesions, particularly for external applications. The products made with bacterial cellulose with the micro-fibrillar component when implanted in the organism are not biotransformed and do not promote remodeling, differing from nanocellulose, which are easily biotransformed and remodeling occurs at the implantation site.

The process of the production of bacterial cellulose hydrogel and the separation in two products, hydrogel of the nano-fibrillar fraction and hydrogel of the micro-fibrillar fraction allows the production of hydrogel of nano-fibrillar cellulose and of micro-fibrillar cellulose to be processed in scale for the processing of specific products for to supply the areas of medicine, pharmacotechnics and cosmiatry.

The process of separation and scale production of nano-fibrillar and micro-fibrillar cellulose hydrogel is a low-cost technological innovation in the line of production of technical economic impact that has not yet been consolidated to date.

In view of the current state of the art, it is being proposed this patent application, which aims to obtain a production process, in scale, of bacterial cellulose hydrogel of micro-fibrillar and nano-fibrillar structure and separation of said hydrogel into two phases, micro-fibrilar bacterial cellulose hydrogel and nano-fibrilar bacterial cellulose hydrogel.

The bacterial cellulose used for the production process of the hydrogel is obtained from renewable sources of sugar derived from sugar cane and from other renewable sources such as dairy products and coconut water by biotechnological means through cellulose-producing bacteria and mainly by Gluconoacetobacter hansenii Imspe isolated from regional biodiversity.

The microorganism Gluconoacetobacter hansenii Imspe isolated from regional biodiversity used in the bacterial cellulose production process is characterized as a high productivity strain of nanocellulose.

The scale production process of bacterial cellulose for hydrogel production is obtained in a sealed sterilization and propagation unit, reactors (biodigesters), which aims at greater control in production, tailoring the product to specific applications such as in the areas of medicine, pharmacotechnics and cosmiatry.

The hydrogel, consisting of bacterial cellulose and water is obtained in scale from purified and hydrated bacterial cellulose with high concentration of micro and nano fibrils with a percentage of crystallinity of 89.8% that meets the specificities and requirements of purity and micro crystallinity suitable for the production of different products for application in the areas of medicine, pharmacotechnics and cosmiatry in demand of scale.

The process of obtaining the hydrogel consists of hydration, mechanical homogenization of purified bacterial cellulose and adjustment of the hydration with distilled apyrogenic water to specific concentration levels of the hydrogel.

The separation in two fractions of hydrogel, one with a higher concentration of nano-fibrillar bacterial cellulose and another with a higher concentration of micro-fibrillar bacterial cellulose, aims to produce bacterial cellulose hydrogel with specific fractions of nano-fibrillar or micro-fibrillar cellulose suitable for the production of products with high specificity for applications in the areas of medicine, pharmacotechnics and cosmiatry in scale demand. The process is an innovation of technical, scientific and economic impact.

The present description relates to the process of scale production of hydrogel from the mass of bacterial cellulose obtained from renewable sources of sugar, particularly sugar cane derivatives, as well as other renewable sources such as dairy derivatives and coconut water biotechnology through the propagation of cellulose-producing bacteria in reactors (biodigesters), particularly the Gluconoacetobacter hansenii Imspe, isolated from the regional biodiversity, purified and hydrated for the production of the hydrogel for direct application and the production of products for multiple applications, particularly in the areas of medicine, pharmacotechnics and cosmiatry. The scale production process of bacterial cellulose hydrogel obtained by the propagation of cellulose-producing bacteria in biodigesters, particularly the Gluconoacetobacter hansenii Imspe in a sealed sterilization and propagation unit, biodigesters and the hydration and mechanical homogenization of the purified cellulosic mass results in hydrogel scale production with controlled hydration.

The direct production of scale bacterial cellulose hydrogel by means of the hydration and mechanical homogenization of the nano-fibrils and micro-fibrils of the purified cellulosic mass for direct application and the production of products for application in the areas of medicine, pharmacotechnics and cosmiatry is an innovation with impact in the industrial and economic scope.

The final product, purified bacterial cellulose hydrogel is produced by the hydration and mechanical homogenization of the purified cellulosic mass consisting of nano-fibrils and micro-fibrils.

The structured hydrogel of nano and micro-fibrils of bacterial cellulose is separated into two phases by means of centrifugal filtration, obtaining a nano-fibrillar and another micro-fibrillar fraction.

The hydrogel structured with the two micro-fibrillar and nano-fibrillar phases as well as the hydrogel consisting of the micro-fibrillar and nano-fibrillar phases can be applied directly or be used for the production of products for applications in the areas of medicine, pharmacotechnics and cosmiatry.

The production of hydrogel in scale from bacterial cellulose obtained from sugars from renewable sources via biotechnology by celulose-producing bacteria and particularly by the Gluconoacetobacter hansenii Imspe isolated from regional biodiversity in a sealed sterilization and propagation unit is a controlled, safe production process that allows the production of structured hydrogels in micro- and nano-fibrils or of nano- and micro-fibrils alone.

The described production process is an innovation of technical and scientific impact of market interest due to the multiplicity and amplitude of its applications in the biological area, particularly in the areas of medicine, pharmacotechnics and cosmiatry.

### SUMMARY OF THE DISCLOSURE

The process proposed in this application consists of the production, in scale, of bacterial cellulose hydrogel produced from sugars from biotechnological renewable sources by cellulose-producing bacteria and particularly by the Gluconoacetobacter hansenii Imspe, isolated from regional biodiversity, in a sealed unit of sterilization and propagation.

The production of the hydrogel is obtained by the hydration and mechanical homogenization of the purified cellulosic mass consisting of micro- and nano-fibrillar structure.

The production of hydrogel in scale from purified bacterial cellulose consisting of micro and nano-fibrillar structure is an impact innovation of market interest due to the multiplicity and amplitude of its applications in the biological area, particularly in the areas of medicine, pharmacotechnics and cosmiatry.

The production of bacterial cellulose hydrogel of micro- and nano-fibrillar structure is obtained by hydration and mechanical homogenization of the purified cellulosic mass, the hydrogel of nano- and micro-fibrillar structure is separated by centrifugal filtration in two phases, hydrogel of nano-fibrillar structure and hydrogel of micro-fibrillar structure.

The scale production of bacterial cellulose hydrogel in two phases, hydrogel of nano-fibrillar structure and hydrogel of micro-fibrillar structure is an innovation of scientific and technical impact of market interest because it broadens the scientific investigation and the use of bacterial cellulose hydrogel micro fibril and nano-fibrillar as a matrix in the production of films, sponges and composites for application in biology, particularly in the areas of medicine, pharmacotechnics and cosmiatry.

### BRIEF DESCRIPTION OF THE FIGURE

This patent application will be described in detail with reference to the FIGURE, where a flowchart demonstrating the process of obtaining the treated hydrogel is shown.

### DETAILED DESCRIPTION OF THE DISCLOSURE

In the present process, the purified bacterial cellulose is obtained by biotechnological means by the propagation of cellulose-producing bacteria and particularly by the Gluconoacetobacter hansenii LMSPE, isolated from regional biodiversity, into a sealed sterilization and propagation unit, such as that discussed in the patent document BR 10 2014 027203 8, of the same holder which is about “PRODUCTION AND PURIFICATION PROCESS IN SCALE OF BACTERIAL CELLULOSE OBTAINED BY THE POLYMERIZATION OF GLUCOSE FROM SUGARS OF RENEWABLE SOURCES VIA BIOTECHNOLOGY BY THE SPREAD OF GLUCONOACETOBACTER HANSENII LMSPE IN REACTORS AND OBTENTION OF PURIFIED CELLULOSE FOR APPLICATION IN THE HEALTH, PHARMACOTECHNICS AND COSMIATRY AREAS”, in medium prepared from sugar cane derivatives such as molasses, concentrated sugar cane syrup, natural juice obtained by the crushing of sugar cane, preferably by the ease of supply among other fruit derivatives such as coconut water or whey derived from milk obtained in the process of dairy products.

The purified bacterial cellulose hydrogel which is a complex of micro-fibrillar cellulose and nano-fibrillar cellulose which is obtained by the mechanical hydration and homogenization of the cellulosic complex.

The process of production of bacterial cellulose hydrogel is divided into two phases: Phase 1, Initiation of the process consisting of the hydration and homogenization of the purified cellulosic mass structured in micro-fibrillar and nano-fibrillar cellulose; and Phase 2, separation process of micro-fibrillar cellulose hydrogel and nano-fibrillar cellulose hydrogel fractions. The process is shown in the FIGURE, where the flowchart F presents the blocks referring to the various steps of the respective processes, where:
F1—represents the beginning of the process, which starts with the raw material, which is defined by purified bacterial cellulose constituted of the components, micro-fibrillar cellulose and nano-fibrillar cellulose;
F2—represents the steps of Hydration (indicated schematically as “H1”) and mechanical homogenization (indicated schematically as “H2”) of the bacterial cellulose, steps which are carried out in an industrial blender;
F3—Obtaining the mixed hydrogel consisting of micro-fibrillar and nano-fibrillar cellulose, which is shown schematically in flowchart F through reference “A”;
F4—Separation process of the mixed hydrogel consisting of micro fibrillar and nano-fibrillar cellulose, obtained in F3 by centrifugal filtration in a blender with a “mesh” screen of separation;
F5—End—Obtention of the products which are: micro-fibrillar cellulose hydrogel, represented by the reference “B” and nano-fibrillar cellulose hydrogel, represented by reference “C”.
B) Micro-fibrillar bacterial cellulose hydrogel, internal phase of the screen, “mesh”; and
C) Nano-fibrillar bacterial cellulose hydrogel, external phase of the screen, “mesh”.

As it should be clear, the process in question leads to the generation of three different products, since in addition to the two versions of the hydrogel: B) micro-fibrillar and C) nano-fibrillar, there is also the mixed hydrogel (“A”) from step F3 of flowchart F.

The cycle of processes used for the production of purified bacterial cellulose hydrogel, structured by synthesis in the micro-fibrillar and nano-fibrillar fractions results in a product with multiple applications as film and composite production for application in the areas of medicine, pharmacotechnics and cosmiatry.

The process of separation and production in scale of nano-fibrillar bacterial cellulose hydrogel and micro-fibrillar bacterial cellulose hydrogel results in obtaining two products, a) Micro-fibrillar bacterial cellulose hydrogel (indicated as “B”) and b) Nano-fibrillar bacterial cellulose hydrogel (indicated as “C”), the scale obtaining of bacterial cellulose hydrogel in separate phases, micro-fibrillar and nano-fibrillar is an innovation of technical and economic impact.

The production of micro-fibrillar bacterial cellulose hydrogel and nano-fibrillar bacterial cellulose hydrogel is a technical innovation with scientific and economic impact by the amplitude of the production of new products using in isolation micro-fibrillar bacterial cellulose hydrogel and nano-fibrillar bacterial cellulose hydrogel for biological application, particularly in the areas of medicine, pharmacotechnics and cosmiatry.

The purified, structured bacterial cellulose mass of nano- and micro-fibrils employed in the present process is hydrated in the proportion of 0.5 to 2.0, part of bacterial cellulose, dry weight of 99.5 to 98.00; part of apyrogenic distilled water and homogenized in an industrial blender to obtain the hydrogel with corresponding concentration. Water can be added to the hydrogel to adjust specific concentrations.

The Hydrogel proposed here, mixed in the two nano- and micro-fibrillar fractions or in a single fraction, nano- or micro-fibrillar can be used as the carrier product of medicaments in suspension, in solution or with nano-capsules in suspension for controlled release of medicaments; as fillers “bulking agent” for the treatment of urinary incontinence, vesico-ureteral reflux, gastroesophageal reflux, fecal incontinence, critical defects of bone and cartilage tissue, filling and modeler in plastic surgery; for the production of films for specific applications, among which is the treatment of burns, varicose ulcers, pressure ulcers, arterial prostheses, venous, myringoplasty, screens or meshes for the treatment of hernias; matrix for the production of composites having as reinforcement different salts such as tricalcium phosphate, hydroxyapatite and calcium hydroxide for applications in the areas of traumatology and orthopedics; as a matrix for the production of sponges, spongy composites and solid composites for the production of plates and screws for application in the areas of health, pharmacotechnics and cosmiatry.

What is claimed is:

1. A scale production process of purified bacterial cellulose hydrogel obtained by the polymerization of glucose from sugars of renewable sources via biotechnology by the spread of cellulose-producing bacteria, particularly gluconoacetobacter hansenii Imspe, in reactors for application in the health, pharmacotechnical and cosmetry areas, characterized by starting from the raw material constituted of purified bacterial cellulose structured in nano-fibrillar and micro-fibrillar cellulose obtained by biotechnological synthesis—block (F1) and processed to obtain hydrogel following the following steps: a) hydration (H1) of the cellulosic mass and b) homogenization (H2) block (F2), obtaining mixed hydrogel (A)—block (F3); c) filtration—block (F4); d) obtaining two fractions of hydrogel, which were: nano-fibrillar bacterial cellulose hydrogel (B) and micro-fibrillar bacterial cellulose hydrogel (C)—block(F5).

2. The scale production process of purified bacterial cellulose hydrogel obtained by the polymerization of glucose from sugars of renewable sources via biotechnology by the spread of cellulose-producing bacteria, particularly gluconoacetobacter hansenii Imspe, in reactors for application in the health, pharmacotechnical and cosmetry areas, according to claim 1, characterized in that the purified bacterial cellulose mass is structured in the synthesis of nano- and micro-fibrils, hydrated in the proportion of 0.5 to 2.0, part of bacterial cellulose, dry weight for 99.5 to 98.00; part of distilled apyrogenic water and homogenized in industrial blender.

3. The scale production process of purified bacterial cellulose hydrogel obtained by the polymerization of glucose from sugars of renewable sources via biotechnology by the spread of cellulose-producing bacteria, particularly gluconoacetobacter hansenii Imspe, in reactors for application in the health, pharmacotechnical and cosmetry areas, according to claim 1, characterized by homogenized or structured purified bacterial cellulose hydrogel of nano- and micro-fibrils, hydrated in the proportion of 0.5 to 2.0, part of bacterial cellulose, dry weight for 99.5 to 98.00; part of distilled apyrogenic water, hydrogel of the purified bacterial cellulose mass, structured of nano- and micro-fibrils.

4. The scale production process of purified bacterial cellulose hydrogel obtained by the polymerization of glucose from sugars of renewable sources via biotechnology by the spread of cellulose-producing bacteria, particularly gluconoacetobacter hansenii Imspe, in reactors for application in the health, pharmacotechnical and cosmetry areas, according to claim 1, characterized by the use of hydrated and homogenized bacterial cellulose polymeric mass, micro-fibrillar and nano-fibrillar bacterial cellulose hydrogel, filtered by centrifugal force in blender with screen, “mesh” for separation into two phases: being the micro-fibrillar bacterial cellulose hydrogel; and the nano-fibrillar bacterial cellulose hydrogel.

5. The scale production process of purified bacterial cellulose hydrogel obtained by the polymerization of glucose from sugars of renewable sources via biotechnology by the spread of cellulose-producing bacteria, particularly gluconoacetobacter hansenii Imspe, in reactors for application in the health, pharmacotechnical and cosmetry areas, according to claim 4, characterized in that it allows obtaining two fractions of hydrogel, where one of these fractions is the micro-fibrillar bacterial cellulose hydrogel, while the other fraction is the nano-fibrillar bacterial cellulose hydrogel for use as carriers products of medicaments in solution, suspension or nano-capsules for controlled release of medicaments.

6. The scale production process of purified bacterial cellulose hydrogel obtained by the polymerization of glucose from sugars of renewable sources via biotechnology by the spread of cellulose-producing bacteria, particularly gluconoacetobacter hansenii Imspe, in reactors for application in the health, pharmacotechnical and cosmetry areas, according to claim 4, characterized in that it allows obtaining two fractions of hydrogel, where one of these fractions is the micro-fibrillar bacterial cellulose hydrogel, while the other fraction is the nano-fibrillar bacterial cellulose hydrogel for use as fillers “bulking agent” for the treatment of urinary incontinence, vesico-ureteral reflux, gastroesophageal reflux, fecal incontinence, critical defects of bone and cartilaginous tissue, fill and modeler in plastic surgery.

7. The scale production process of purified bacterial cellulose hydrogel obtained by the polymerization of glucose from sugars of renewable sources via biotechnology by the spread of cellulose-producing bacteria, particularly gluconoacetobacter hansenii Imspe, in reactors for application in the health, pharmacotechnical and cosmetry areas, according to claim 4, characterized in that it allows obtaining two fractions of hydrogel, where one of these fractions is the micro-fibrillar bacterial cellulose hydrogel; where the other of these two fractions is the nano-fibrillar bacterial cellulose hydrogel used for the production of films for general applications, among which specific surgical dressings for the treatment of burns, varicose ulcers and pressure ulcers, myringoplasty, arterial, venous patches, common bile duct, nail prostheses and mesh meshes for reinforcement in the treatment of hernias.

8. The scale production process of purified bacterial cellulose hydrogel obtained by the polymerization of glucose from sugars of renewable sources via biotechnology by the spread of cellulose-producing bacteria, particularly gluconoacetobacter hansenii Imspe, in reactors for application in the health, pharmacotechnical and cosmetry areas, according to claim 4, characterized in that it allows obtaining two fractions of hydrogel, where one of the fractions is the micro-fibrillar bacterial cellulose hydrogel; where another of these two fractions is the nano-fibrillar bacterial cellulose hydrogel, application of the mixed hydrogel, in the two nano- and micro-fibrillar fractions as matrix for the production of composites having as reinforcement different salts as tricalcicic phosphate, hydroxyapatite and calcium hydroxide for applications in the areas of traumatology and orthopedics, bucomaxillofacial and dentistry.

9. The scale production process of purified bacterial cellulose hydrogel obtained by the polymerization of glucose from sugars of renewable sources via biotechnology by the spread of cellulose-producing bacteria, particularly gluconoacetobacter hansenii Imspe, in reactors for application in the health, pharmacotechnical and cosmetry areas, according to claim 4, characterized in that it allows obtaining two fractions of hydrogel, where one of the fractions is the micro-fibrillar bacterial cellulose hydrogel; where another of these two fractions is the nano-fibrillar bacterial cellulose hydrogel, using each fraction alone, micro-fibrillar bacterial cellulose hydrogel and nano-fibrillar bacterial cellulose hydrogel as a matrix for the production of composites having as reinforcement tricalcicic phosphate, hydroxyapatite and calcium hydroxide for applications in the areas of traumatology, orthopedics, bucomaxillofacial and dentistry and plastic surgery.

10. The scale production process of purified bacterial cellulose hydrogel obtained by the polymerization of glucose from sugars of renewable sources via biotechnology by the spread of cellulose-producing bacteria, particularly gluconoacetobacter hansenii Imspe, in reactors for application in the health, pharmacotechnical and cosmetry areas, according to claim 4, characterized in that it allows obtaining two fractions of hydrogel, where one of the fractions is the micro-fibrillar bacterial cellulose hydrogel; while the other of these fractions is the nano-fibrillar bacterial cellulose hydrogel; application of the mixed hydrogel in the two nano and micro-fibrillar fractions as a matrix for the production of sponges for application in the areas of health, medicine, pharmacotechnics and cosmiatry.

11. The scale production process of purified bacterial cellulose hydrogel obtained by the polymerization of glucose from sugars of renewable sources via biotechnology by the spread of cellulose-producing bacteria, particularly gluconoacetobacter hansenii Imspe, in reactors for application in the health, pharmacotechnical and cosmetry areas, according to claim 4, characterized in that it allows obtaining two fractions of hydrogel, where one of the fractions is the micro-fibrillar bacterial cellulose hydrogel, while the other of these fractions is the nano-fibrillar bacterial cellulose hydrogel, used alone micro-fibrillar bacterial cellulose hydrogel and nano-fibrillar bacterial cellulose hydrogel as a matrix for the production of sponges; micro-fibrillar bacterial cellulose sponges and nano-fibrillar bacterial cellulose sponges for application in the areas of health, medicine, pharmacotechnics, cosmiatry and plastic surgery.

12. The scale production process of purified bacterial cellulose hydrogel obtained by the polymerization of glucose from sugars of renewable sources via biotechnology by the spread of cellulose-producing bacteria, particularly gluconoacetobacter hansenii Imspe, in reactors for application in the health, pharmacotechnical and cosmetry areas, according to claim 4, characterized in that it allows obtaining two fractions of hydrogel, where one of the fractions is the micro-fibrillar bacterial cellulose hydrogel, while the other of these fractions is the nano-fibrillar bacterial cellulose hydrogel, being obtained a third fraction of Hydrogel that is the mixed hydrogel; wherein the mixed hydrogel, or the nano-fibrillar or micro-fibrillar fractions can be used alone or in combination as matrices with reinforcements of organic salts for the production of solid or spongy composites for application such as plaques, rods, screws and fills in the areas of health, traumatology, orthopedics, bucomaxillofacial and dentistry and plastic surgery.
